(19) United States
(12) Patent Application Publication
(10) Pub. No.: US 2022/0339124 A1
(43) Pub. Date: Oct. 27, 2022

(54) INTRANASAL PHARMACEUTICAL COMPOSITIONS OF CYCLOBENZAPRINE

(71) Applicant: CADILA HEALTHCARE LIMITED, Ahmedabad, Gujarat (IN)

(72) Inventors: Kannan Essakimuthu MUTHAIYYAN, Ahmedabad, Gujarat (IN); Ritu Nitin LADDHA, Ahmedabad, Gujarat (IN); Harish PAWAR, Ahmedabad, Gujarat (IN); Shailesh Arvindbhai PATEL, Ahmedabad, Gujarat (IN); Jay Shantilal KOTHARI, Pennington, NJ (US)

(21) Appl. No.: 17/765,030

(22) PCT Filed: Sep. 30, 2020
(86) PCT No.: PCT/IB2020/059144

§ 371 Date: Mar. 30, 2022

(30) Foreign Application Priority Data
Sep. 30, 2019 (IN) 201921039492

(51) Int. Cl. A61K 31/135; A61K 9/00; A61K 9/08

(52) U.S. Cl. CPC A61K 31/135; A61K 9/0043; A61K 9/08; A61K 9/008; A61M 2210/0618; A61H 35/04

(57) ABSTRACT

The present invention relates to intranasal pharmaceutical compositions comprising cyclobenzaprine or salts thereof for the treatment or management of muscle spasms in musculoskeletal diseases and/or painful physical symptoms in neurological diseases. In particular, the invention relates to intranasal pharmaceutical compositions comprising cyclobenzaprine or salts thereof and one or more pharmaceutically acceptable excipients.

### FIELD OF THE INVENTION

The present invention provides intranasal pharmaceutical compositions comprising cyclobenzaprine or pharmaceutically acceptable salts thereof for the treatment or management of muscle spasms in musculoskeletal diseases and/or painful physical symptoms in neurological diseases. In particular, the invention relates to intranasal pharmaceutical compositions of cyclobenzaprine or pharmaceutically acceptable salts thereof and one or more pharmaceutically acceptable excipients.

The intranasal pharmaceutical compositions of the present invention, when administered to a mammal, the onset of action of the drug is provided rapidly to effectively treat or manage the muscle spasms in musculoskeletal diseases and/or painful physical symptoms in neurological diseases.

### BACKGROUND OF THE INVENTION

Cyclobenzaprine or 3-(5H-Dibenzo[a,d]cyclohepten-5-ylidene)-N,N-dimethyl-1-propanamine, is represented by the chemical formula:

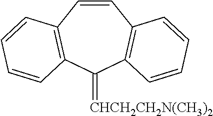

Cyclobenzaprine was first synthesized in 1961. [Villani, F. J., et al., “Dialkylaminoalkyl derivatives of 10,11-dihydro-511-dibenzo a,d cycloheptene and related compounds,” J. Med. Pharm. Chem. 5:373-383 (1962)]. Cyclobenzaprine is a skeletal muscle relaxant and is sold in the United States as 5 mg and 10 mg tablets under the trade name, FLEXERIL® (cyclobenzaprine hydrochloride) by Janssen Research and Development LLC. Its pharmaceutical action comprises of relieving the skeletal muscle spasm without adversely interfering with the muscle function. It is also approved as 15 mg and 30 mg extended release capsules and is sold in the United States under the trade name, AMRIX® (cyclobenzaprine hydrochloride).

Cyclobenzaprine hydrochloride is well absorbed in humans after oral administration, but there is a large inter-subject variation in plasma levels of the individuals to whom the drug has been administered. Cyclobenzaprine hydrochloride is extensively metabolized in humans. In the study with 14C-labelled drug, about 1% of the dose was excreted in the urine as unchanged cyclobenzaprine hydrochloride. Cyclobenzaprine is eliminated quite slowly, with an effective half-life of 18 hours (range 8-37 hours; n=18); plasma clearance is 0.7 L/min. Extensive work has been done on the oral dosage of the cyclobenzaprine.

International Publication No. (PCT) WO 99/18937 is directed to a composition comprising a pharmaceutically effective amount of cyclobenzaprine and calcium phosphate dibasic hydrous, wherein the tablet releases most of the active component within an hour.

U.S. Pat. Nos. 7,387,793; 7,544,372, and U.S. Patent Publication Nos. 2008/0124398; 2009/0017126; 2009/0017127; and 2009/0148532 disclose multi-particulate pharmaceutical dosage forms of a skeletal muscle relaxant for once a day administration comprising a population of extended release beads.

U.S. Patent Publication No. 20140024720 discloses a pharmaceutical composition comprising 5 to 20% w/v of cyclobenzaprine hydrochloride, acceptable preservatives, buffer solutions, muco-adhesive and absorption enhancer substances, wherein the pharmaceutical composition has a muscle relaxant activity and is suitable for intranasal administration. The patent application compares nasal spray with the oral dosage form comprising similar concentration of cyclobenzaprine in both nasal spray and oral solution.

U.S. Patent Publication No. 20140171515 discloses a composition comprising cyclobenzaprine and a basifying agent, wherein the composition is suitable for transmucosal absorption. The composition disclosed in the art is specifically suitable for sublingual administration.

U.S. Pat. No. 9,918,948 specifically describes a method for treating post-traumatic stress disorder (PTSD) following a traumatic event by administering to a human in need of such treatment a pharmaceutical composition comprising cyclobenzaprine in a therapeutically effective amount and a therapeutically effective carrier, wherein such treatment eliminates or ameliorates the PTSD.

U.S. Pat. No. 6,395,788 discloses a method for treating a sleep disturbance comprising the step of administering to a human in need of treatment for such sleep disturbance a composition comprising cyclobenzaprine in an amount of less than 5 mg/day, wherein the human is not suffering from the fibromyalgia syndrome. This art specifically studied the oral dosage form for the treatment of sleep disturbances.

However, despite the broad therapeutic usefulness of cyclobenzaprine, cyclobenzaprine is absorbed slowly into the blood stream after oral administration and should be taken approximately one to two hours before an effect is desired. Further, orally administered cyclobenzaprine has several side-effects which may hinder the routine activities of the subject to whom the dose was administered, e.g., drowsiness and lack of alertness.

Hence, there is an unmet need to develop a dosage form which provides an early onset of action at low dose and meeting the unmet requirements in the treatment of the muscle spasms in musculoskeletal diseases and painful physical symptoms in neurological diseases.

The present invention relates to an intranasal delivery of cyclobenzaprine or pharmaceutically acceptable salts thereof, which has a number of significant and unexpected advantages over the oral dosage form. The advantages of this intranasal route of administration are rapid onset of action compared to oral route of administration, improved safety, and reduced exposure variability resulting in reduced incidence of adverse side effects.

### SUMMARY OF THE INVENTION

In one general aspect, the present invention relates to intranasal pharmaceutical compositions comprising from about 0.01 mg/dose to about 2.5 mg/dose of cyclobenzaprine or pharmaceutically acceptable salts thereof and one or more pharmaceutically acceptable excipients, wherein the intranasal composition provides an improved pharmacokinetic profile over the administration via oral route.

In one general aspect, the present invention relates to intranasal pharmaceutical compositions comprising from about 0.01 mg/dose to about 2.5 mg/dose of cyclobenzaprine or pharmaceutically acceptable salts thereof and one or more pharmaceutically acceptable excipients, wherein the intranasal pharmaceutical composition provides a Cₘₐₓ of cyclobenzaprine or salts thereof from about 5 ng/mL to about 100 ng/mL at a tₘₐₓ of less than 120 minutes after administration.

In more detailed aspect, the present invention relates to intranasal pharmaceutical compositions comprising from about 0.01 mg/dose to about 2.5 mg/dose of cyclobenzaprine or pharmaceutically acceptable salts thereof and one or more pharmaceutically acceptable excipients, wherein the nasal composition provides a Cₘₐₓ of cyclobenzaprine or salts thereof from about 5 ng/mL to about 100 ng/mL at a tₘₐₓ of less than 60 minutes after administration.

In another detailed aspect, the present invention relates to intranasal pharmaceutical compositions comprising from about 0.01 mg/dose to about 2.5 mg/dose of cyclobenzaprine or pharmaceutically acceptable salts thereof and one or more pharmaceutically acceptable excipients having a pH of the composition from about 5 to about 7, wherein the nasal composition provides a Cₘₐₓ of cyclobenzaprine or salts thereof from about 5 ng/mL to about 100 ng/mL at a tₘₐₓ of less than 30 minutes after administration.

Embodiments of the pharmaceutical composition may include one or more of the following features. For example, the pharmaceutical composition may include one or more pharmaceutically acceptable excipients. The pharmaceutically acceptable excipient may include a preservative, an antioxidant, a buffering agent, a mucoadhesive agent, an acidifying agent, an alkalizing agent, a sweetener, a taste masking agent, a humectant, a chelating agent, a tonicity agent, a surfactant, and the like.

In one embodiment, the pharmaceutically acceptable excipient may include agents which may help in enhancing bioavailability of the pharmaceutical composition, i.e., bioavailability enhancers.

In another general aspect, the present invention provides a process for the preparation of an intranasal pharmaceutical composition comprising from about 0.01 mg/dose to about 2.5 mg/dose of cyclobenzaprine or pharmaceutically acceptable salts thereof and one or more pharmaceutically acceptable excipients, the process comprising:

i) mixing one or more pharmaceutically acceptable excipients with water for injection under continuous stirring to form a solution;

ii) dissolving cyclobenzaprine or pharmaceutically acceptable salts thereof into the solution of step i) under continuous stirring; and

iii) filtering the solution of step ii) through a microfilter and packed in a suitable container.

In another general aspect, there is provided a method of treating or managing the muscle spasm in musculoskeletal diseases and/or painful physical symptoms in neurological diseases, the method comprising administering to a human being in need thereof, an intranasal pharmaceutical composition comprising from about 0.01 mg/dose to about 2.5 mg/dose of cyclobenzaprine or pharmaceutically acceptable salts thereof and one or more pharmaceutically acceptable excipients.

In yet another general aspect, the invention relates to a pharmaceutical kit comprising an intranasal pharmaceutical composition comprising from about 0.01 mg/dose to about 2.5 mg/dose of cyclobenzaprine or pharmaceutically acceptable salts thereof and one or more pharmaceutically acceptable excipients, and a nasal pump device for accurate delivery of said intranasal pharmaceutical composition.

In another general aspect, the intranasal pharmaceutical composition of present invention is administered in a nasal pump device that delivers about 0.01 mg/dose to about 2.5 mg/dose of cyclobenzaprine or pharmaceutically acceptable salts thereof by a single or multiple actuation(s).

Additional objects and advantages of the invention will be set forth in part in the description which follows, and in part will be obvious from the description, or may be learned by practice of the invention.

### DESCRIPTION OF THE INVENTION

Technical and scientific terms used herein have the meaning commonly understood by one of skill in the art to which the present invention pertains, unless otherwise defined.

Spasms can affect many different types of muscles in the body, leading to many different symptoms. Spasms of skeletal muscles are most common and are often due to overuse and muscle fatigue, dehydration, electrolyte abnormalities or any other underlying diseases. Most of the time spasm occurs abruptly, painful and usually short-lived. It may be relieved by gently stretching the muscle. However, sometimes muscle spasms are especially very painful, do not resolve and have recurrence. Therefore, require immediate medical care. In such condition of sudden and sharp pain, dosage form with rapid onset of action and having patient compliance is needed.

Intranasal drug delivery is considered as a promising route of drug delivery system for treating such sudden and sharp pain due to muscle spasm. A large number of factors influence the therapeutic efficacy as well as toxicity of nasally administered drug product. The most important advantages of intranasal drug delivery systems are the presence of large surface area, porous endothelial membrane, high total blood flow, the avoidance of first-pass metabolism, and ready accessibility. Drugs are cleared rapidly from the nasal cavity after intranasal administration, resulting in rapid systemic drug absorption. Therefore, provides an early onset of action and providing immediate relief in case of sudden and sharp pain due to muscle spasm.

Currently, cyclobenzaprine is available as oral tablets in 5 mg and 10 mg strengths. In the oral drug delivery, the systemic bioavailability of a drug is often subjected to variable gastrointestinal transit time and biotransformation in the liver by “first pass metabolism”. Hence, to achieve the desired therapeutic effect the dose needs to be increased which leads to undesired side effects. The most prominent one is the drowsiness, which may limit the movement of the person and may hinder the routine activities.

Accordingly, it would be desirable to improve the bioavailability of cyclobenzaprine or salts thereof and to reduce the necessary daily dose of cyclobenzaprine or salts thereof.

Therefore, it would be advantageous to have a composition of cyclobenzaprine or salts thereof which increases the drug's bioavailability and thus could be dosed at lower doses and provides the efficacy benefits of a higher dose. Further, such formulation could provide rapid onset and consistent action using a lower dose and reducing drug interactions and side-effects because of more consistent delivery and improved bioavailability. For example, a formulation which is, say 50% more bioavailable could be dosed at 2.5 mg and provide the same systemic exposure as currently available formulations when dosed at 5 mg.

The inventors of the present invention have discovered that when cyclobenzaprine or salts thereof is formulated along with one or more pharmaceutically acceptable excipients, the intranasal composition so formed crosses the nasal physiological barrier effectively and provided effective and early relief to patients at significantly low doses.

Improved pharmacokinetics means an enhancement of pharmacokinetic profile is achieved as measured, for example, standard pharmacokinetic parameters such as time to reach maximum plasma concentration (tₘₐₓ), and the magnitude of maximum plasma concentration (Cₘₐₓ), etc. The measurement of pharmacokinetic parameters and determination of minimally effective concentrations are routinely performed in the art. Values obtained are deemed to be enhanced in comparison with a standard route of administration, for example, oral administration. The inventors of the present invention surprisingly found that when cyclobenzaprine or salts thereof is given in the intranasal composition along with and one or more pharmaceutically acceptable excipients in comparison to the commercially available immediate release oral dosage form of cyclobenzaprine, i.e., tablets, it provides maximum plasma concentration in less than one fourth of the time required in case of immediate release oral dosage form, i.e., tablets.

In one preferred embodiment, the present invention provides intranasal pharmaceutical compositions comprising from about 0.01 mg/dose to about 2.5 mg/dose of cyclobenzaprine or pharmaceutically acceptable salts thereof and one or more pharmaceutically acceptable excipients, wherein the nasal composition provides a Cₘₐₓ of cyclobenzaprine or salts thereof from about 5 ng/mL to about 100 ng/mL at a tₘₐₓ of less than 60 minutes after administration.

In one particular embodiment the cyclobenzaprine is administered at a very low dose to minimize side effects observed at higher doses. The doses may include about 0.010 mg/dose, 0.015 mg/dose, 0.020 mg/dose, 0.025 mg/dose, 0.030 mg/dose, 0.035 mg/dose, 0.040 mg/dose, 0.045 mg/dose, 0.050 mg/dose, 0.055 mg/dose, 0.060 mg/dose, 0.065 mg/dose, 0.070 mg/dose, 0.075 mg/dose, 0.080 mg/dose, 0.085 mg/dose, 0.090 mg/dose, 0.095 mg/dose, 0.100 mg/dose, 0.105 mg/dose, 0.110 mg/dose, 0.115 mg/dose, 0.120 mg/dose, 0.125 mg/dose, 0.130 mg/dose, 0.135 mg/dose, 0.140 mg/dose, 0.145 mg/dose, 0.150 mg/dose, 0.155 mg/dose, 0.160 mg/dose, 0.165 mg/dose, 0.170 mg/dose, 0.175 mg/dose, 0.180 mg/dose, 0.185 mg/dose, 0.190 mg/dose, 0.195 mg/dose, 0.200 mg/dose, 0.205 mg/dose, 0.210 mg/dose, 0.215 mg/dose, 0.220 mg/dose, 0.225 mg/dose, 0.230 mg/dose, 0.235 mg/dose, 0.240 mg/dose, 0.245 mg/dose, 0.250 mg/dose, 0.255 mg/dose, 0.260 mg/dose, 0.265 mg/dose, 0.270 mg/dose, 0.275 mg/dose, 0.280 mg/dose, 0.285 mg/dose, 0.290 mg/dose, 0.295 mg/dose, 0.300 mg/dose, 0.305 mg/dose, 0.310 mg/dose, 0.315 mg/dose, 0.320 mg/dose, 0.325 mg/dose, 0.330 mg/dose, 0.335 mg/dose, 0.340 mg/dose, 0.345 mg/dose, 0.350 mg/dose, 0.355 mg/dose, 0.360 mg/dose, 0.365 mg/dose, 0.370 mg/dose, 0.375 mg/dose, 0.380 mg/dose, 0.385 mg/dose, 0.390 mg/dose, 0.395 mg/dose, 0.400 mg/dose, 0.405 mg/dose, 0.410 mg/dose, 0.415 mg/dose, 0.420 mg/dose, 0.425 mg/dose, 0.430 mg/dose, 0.435 mg/dose, 0.440 mg/dose, 0.445 mg/dose, 0.450 mg/dose, 0.455 mg/dose, 0.460 mg/dose, 0.465 mg/dose, 0.470 mg/dose, 0.475 mg/dose, 0.480 mg/dose, 0.485 mg/dose, 0.490 mg/dose, 0.495 mg/dose, 0.500 mg/dose, 0.505 mg/dose, 0.510 mg/dose, 0.515 mg/dose, 0.520 mg/dose, 0.525 mg/dose, 0.530 mg/dose, 0.535 mg/dose, 0.540 mg/dose, 0.545 mg/dose, 0.550 mg/dose, 0.555 mg/dose, 0.560 mg/dose, 0.565 mg/dose, 0.570 mg/dose, 0.575 mg/dose, 0.580 mg/dose, 0.585 mg/dose, 0.590 mg/dose, 0.595 mg/dose, 0.600 mg/dose, 0.605 mg/dose, 0.610 mg/dose, 0.615 mg/dose, 0.620 mg/dose, 0.625 mg/dose, 0.630 mg/dose, 0.635 mg/dose, 0.640 mg/dose, 0.645 mg/dose, 0.650 mg/dose, 0.655 mg/dose, 0.660 mg/dose, 0.665 mg/dose, 0.670 mg/dose, 0.675 mg/dose, 0.680 mg/dose, 0.685 mg/dose, 0.690 mg/dose, 0.695 mg/dose, 0.700 mg/dose, 0.705 mg/dose, 0.710 mg/dose, 0.715 mg/dose, 0.720 mg/dose, 0.725 mg/dose, 0.730 mg/dose, 0.735 mg/dose, 0.740 mg/dose, 0.745 mg/dose, 0.750 mg/dose, 0.755 mg/dose, 0.760 mg/dose, 0.765 mg/dose, 0.770 mg/dose, 0.775 mg/dose, 0.780 mg/dose, 0.785 mg/dose, 0.790 mg/dose, 0.795 mg/dose, 0.800 mg/dose, 0.805 mg/dose, 0.810 mg/dose, 0.815 mg/dose, 0.820 mg/dose, 0.825 mg/dose, 0.830 mg/dose, 0.835 mg/dose, 0.840 mg/dose, 0.845 mg/dose, 0.850 mg/dose, 0.855 mg/dose, 0.860 mg/dose, 0.865 mg/dose, 0.870 mg/dose, 0.875 mg/dose, 0.880 mg/dose, 0.885 mg/dose, 0.890 mg/dose, 0.895 mg/dose, 0.900 mg/dose, 0.905 mg/dose, 0.910 mg/dose, 0.915 mg/dose, 0.920 mg/dose, 0.925 mg/dose, 0.930 mg/dose, 0.935 mg/dose, 0.940 mg/dose, 0.945 mg/dose, 0.950 mg/dose, 0.955 mg/dose, 0.960 mg/dose, 0.965 mg/dose, 0.970 mg/dose, 0.975 mg/dose, 0.980 mg/dose, 0.985 mg/dose, 0.990 mg/dose, 0.995 mg/dose, 1.000 mg/dose, 1.005 mg/dose, 1.010 mg/dose, 1.015 mg/dose, 1.020 mg/dose, 1.025 mg/dose, 1.030 mg/dose, 1.035 mg/dose, 1.040 mg/dose, 1.045 mg/dose, 1.050 mg/dose, 1.055 mg/dose, 1.060 mg/dose, 1.065 mg/dose, 1.070 mg/dose, 1.075 mg/dose, 1.080 mg/dose, 1.085 mg/dose, 1.090 mg/dose, 1.095 mg/dose, 1.100 mg/dose, 1.105 mg/dose, 1.110 mg/dose, 1.115 mg/dose, 1.120 mg/dose, 1.125 mg/dose, 1.130 mg/dose, 1.135 mg/dose, 1.140 mg/dose, 1.145 mg/dose, 1.150 mg/dose, 1.155 mg/dose, 1.160 mg/dose, 1.165 mg/dose, 1.170 mg/dose, 1.175 mg/dose, 1.180 mg/dose, 1.185 mg/dose, 1.190 mg/dose, 1.195 mg/dose, 1.200 mg/dose, 1.205 mg/dose, 1.210 mg/dose, 1.215 mg/dose, 1.220 mg/dose, 1.225 mg/dose, 1.230 mg/dose, 1.235 mg/dose, 1.240 mg/dose, 1.245 mg/dose, 1.250 mg/dose, 1.255 mg/dose, 1.260 mg/dose, 1.265 mg/dose, 1.270 mg/dose, 1.275 mg/dose, 1.280 mg/dose, 1.285 mg/dose, 1.290 mg/dose, 1.295 mg/dose, 1.300 mg/dose, 1.305 mg/dose, 1.310 mg/dose, 1.315 mg/dose, 1.320 mg/dose, 1.325 mg/dose, 1.330 mg/dose, 1.335 mg/dose, 1.340 mg/dose, 1.345 mg/dose, 1.350 mg/dose, 1.355 mg/dose, 1.360 mg/dose, 1.365 mg/dose, 1.370 mg/dose, 1.375 mg/dose, 1.380 mg/dose, 1.385 mg/dose, 1.390 mg/dose, 1.395 mg/dose, 1.400 mg/dose, 1.405 mg/dose, 1.410 mg/dose, 1.415 mg/dose, 1.420 mg/dose, 1.425 mg/dose, 1.430 mg/dose, 1.435 mg/dose, 1.440 mg/dose, 1.445 mg/dose, 1.450 mg/dose, 1.455 mg/dose, 1.460 mg/dose, 1.465 mg/dose, 1.470 mg/dose, 1.475 mg/dose, 1.480 mg/dose, 1.485 mg/dose, 1.490 mg/dose, 1.495 mg/dose, 1.500 mg/dose, 1.505 mg/dose, 1.510 mg/dose, 1.515 mg/dose, 1.520 mg/dose, 1.525 mg/dose, 1.530 mg/dose, 1.535 mg/dose, 1.540 mg/dose, 1.545 mg/dose, 1.550 mg/dose, 1.555 mg/dose, 1.560 mg/dose, 1.565 mg/dose, 1.570 mg/dose, 1.575 mg/dose, 1.580 mg/dose, 1.585 mg/dose, 1.590 mg/dose, 1.595 mg/dose, 1.600 mg/dose, 1.605 mg/dose, 1.610 mg/dose, 1.615 mg/dose, 1.620 mg/dose, 1.625 mg/dose, 1.630 mg/dose, 1.635 mg/dose, 1.640 mg/dose, 1.645 mg/dose, 1.650 mg/dose, 1.655 mg/dose, 1.660 mg/dose, 1.665 mg/dose, 1.670 mg/dose, 1.675 mg/dose, 1.680 mg/dose, 1.685 mg/dose, 1.690 mg/dose, 1.695 mg/dose, 1.700 mg/dose, 1.705 mg/dose, 1.710 mg/dose, 1.715 mg/dose, 1.720 mg/dose, 1.725 mg/dose, 1.730 mg/dose, 1.735 mg/dose, 1.740 mg/dose, 1.745 mg/dose, 1.750 mg/dose, 1.755 mg/dose, 1.760 mg/dose, 1.765 mg/dose, 1.770 mg/dose, 1.775 mg/dose, 1.780 mg/dose, 1.785 mg/dose, 1.790 mg/dose, 1.795 mg/dose, 1.800 mg/dose, 1.805 mg/dose, 1.810 mg/dose, 1.815 mg/dose, 1.820 mg/dose, 1.825 mg/dose, 1.830 mg/dose, 1.835 mg/dose, 1.840 mg/dose, 1.845 mg/dose, 1.850 mg/dose, 1.855 mg/dose, 1.860 mg/dose, 1.865 mg/dose, 1.870 mg/dose, 1.875 mg/dose, 1.880 mg/dose, 1.885 mg/dose, 1.890 mg/dose, 1.895 mg/dose, 1.900 mg/dose, 1.905 mg/dose, 1.910 mg/dose, 1.915 mg/dose, 1.920 mg/dose, 1.925 mg/dose, 1.930 mg/dose, 1.935 mg/dose, 1.940 mg/dose, 1.945 mg/dose, 1.950 mg/dose, 1.955 mg/dose, 1.960 mg/dose, 1.965 mg/dose, 1.970 mg/dose, 1.975 mg/dose, 1.980 mg/dose, 1.985 mg/dose, 1.990 mg/dose, 1.995 mg/dose, 2.000 mg/dose, 2.005 mg/dose, 2.010 mg/dose, 2.015 mg/dose, 2.020 mg/dose, 2.025 mg/dose, 2.030 mg/dose, 2.035 mg/dose, 2.040 mg/dose, 2.045 mg/dose, 2.050 mg/dose, 2.055 mg/dose, 2.060 mg/dose, 2.065 mg/dose, 2.070 mg/dose, 2.075 mg/dose, 2.080 mg/dose, 2.085 mg/dose, 2.090 mg/dose, 2.095 mg/dose, 2.100 mg/dose, 2.105 mg/dose, 2.110 mg/dose, 2.115 mg/dose, 2.120 mg/dose, 2.125 mg/dose, 2.130 mg/dose, 2.135 mg/dose, 2.140 mg/dose, 2.145 mg/dose, 2.150 mg/dose, 2.155 mg/dose, 2.160 mg/dose, 2.165 mg/dose, 2.170 mg/dose, 2.175 mg/dose, 2.180 mg/dose, 2.185 mg/dose, 2.190 mg/dose, 2.195 mg/dose, 2.200 mg/dose, 2.205 mg/dose, 2.210 mg/dose, 2.215 mg/dose, 2.220 mg/dose, 2.225 mg/dose, 2.230 mg/dose, 2.235 mg/dose, 2.240 mg/dose, 2.245 mg/dose, 2.250 mg/dose, 2.255 mg/dose, 2.260 mg/dose, 2.265 mg/dose, 2.270 mg/dose, 2.275 mg/dose, 2.280 mg/dose, 2.285 mg/dose, 2.290 mg/dose, 2.295 mg/dose, 2.300 mg/dose, 2.305 mg/dose, 2.310 mg/dose, 2.315 mg/dose, 2.320 mg/dose, 2.325 mg/dose, 2.330 mg/dose, 2.335 mg/dose, 2.340 mg/dose, 2.345 mg/dose, 2.350 mg/dose, 2.355 mg/dose, 2.360 mg/dose, 2.365 mg/dose, 2.370 mg/dose, 2.375 mg/dose, 2.380 mg/dose, 2.385 mg/dose, 2.390 mg/dose, 2.395 mg/dose, 2.400 mg/dose, 2.405 mg/dose, 2.410 mg/dose, 2.415 mg/dose, 2.420 mg/dose 2.420 mg/dose, 2.425 mg/dose, 2.430 mg/dose, 2.435 mg/dose, 2.440 mg/dose, 2.445 mg/dose, 2.450 mg/dose, 2.455 mg/dose, 2.460 mg/dose, 2.465 mg/dose, 2.470 mg/dose, 2.475 mg/dose, 2.480 mg/dose, 2.485 mg/dose, 2.490 mg/dose, 2.495 mg/dose, and 2.500 mg/dose.

In further embodiment, dose may include dosage or amount of drug administered to a person per administration or drug administered to a person over a period of 24 hours.

Surprisingly, the inventors of the present invention have also found that intranasal compositions of cyclobenzaprine or salts thereof have a pH of the composition from about 5 to about 7 and have excellent solubility properties. The composition is acceptable to the general patient population and does not cause unnecessary irritancy as pH of the composition from about 5 to about 7. The nasal mucosal pH is in the range of 5-6.5. Moreover, cyclobenzaprine is hydrophilic drug and this particular property of the drug further hinders the absorption of cyclobenzaprine or salts thereof through the mucous membrane, specifically the nasal mucosa. Also, cyclobenzaprine has a pKa of 8.47 at room temperature. It is known in the art that permeation of drug is greater if the nasal pH is lower than pKa of drug because under such conditions the penetrant molecules exist as unionized species. Hence, inventors of the present invention found that if the pH of the intranasal composition of cyclobenzaprine is maintained from about 5 to about 7, it further enhances the absorption of cyclobenzaprine and enhances the pharmacokinetic profile significantly. Therefore, it further helps in providing the therapeutic effect at low dose and also the onset of action is significantly enhanced.

Therefore, in one preferred embodiment, the present invention provides intranasal pharmaceutical compositions comprising from about 0.01 mg/dose to about 2.5 mg/dose of cyclobenzaprine or pharmaceutically acceptable salts thereof and one or more pharmaceutically acceptable excipients with pH of the composition from about 5 to about 7, wherein the nasal composition provides a Cₘₐₓ of cyclobenzaprine or salts thereof from about 5 ng/ml to about 100 ng/mL at a tₘₐₓ of less than 30 minutes after administration.

In another embodiment, the nasal composition provides a Cₘₐₓ of cyclobenzaprine or salts thereof from about 5 ng/mL to about 100 ng/mL at a tₘₐₓ of less than 15 minutes after administration.

In yet another embodiment, the present invention provides a process for the preparation of an intranasal pharmaceutical composition comprising from about 0.01 mg/dose to about 2.5 mg/dose of cyclobenzaprine or pharmaceutically acceptable salts thereof and one or more pharmaceutically acceptable excipients, the process comprising:

i) mixing one or more pharmaceutically acceptable excipients with water for injection under continuous stirring to form a solution;

ii) dissolving cyclobenzaprine or pharmaceutically acceptable salts thereof into the solution of step i) under continuous stirring; and

iii) filtering the solution of step ii) through a microfilter and pack in a suitable container.

The intranasal pharmaceutical composition of the present invention further comprises one or more pharmaceutically acceptable excipients which may be selected from, but not limited to, preservatives, antioxidants, buffering agents, mucoadhesive agents, acidifying agents, alkalizing agents, sweeteners, taste masking agents, humectants, chelating agents, tonicity agents, surfactants, or mixtures thereof.

In some specific embodiments, the pharmaceutical composition may include agents which may help in enhancing bioavailability of the pharmaceutical composition.

Suitable agents which may act as bioavailability enhancers may be selected from one or more of disodium hydrogen phosphate, disodium hydrogen phosphate dodecahydrate dipotassium hydrogen phosphate, sodium carbonate, sodium bicarbonate, potassium dihydrogen phosphate, calcium carbonate, calcium bicarbonate, sodium dihydrogen phosphate, trisodium phosphate, tripotassium phosphate, potassium carbonate, potassium bicarbonate, potassium acetate, sodium acetate, dipotassium citrate, tripotassium citrate, disodium citrate, trisodium citrate, TRIS buffer, alkyl glycoside or saccharide alkyl ester selected from (1-O-n-Dodecyl-β-D-Maltopyranoside), tridecyl maltoside, sucrose monododecanoate, sucrose monotridecanoate, sucrose monotetradecanoate, L-a-Lysophosphatidylcholine (lysolecithin) (LPC), palmitoyl-DL-carnitine chloride (PCC), N-acety 1-L-cysteine (ACS), amastatin hydrochloride (AMA), bestatin hydrochloride (BES), a-phenylenediamine dihydrochloride (OPD), human serum albumin (HSA), sodium lauryl sulphate, cetylpyridinium chloride, poloxamers, sorbitan and its salts, glycocholic acid and its derivatives like sodium glycodeoxycholate, sodium glycocholate, sodium taurodeoxycholate, cyclodextrin, α,β,γ-cyclodextrin, methylated β-cyclodextrins oleic acid, methyloleate, lauric acid, caprylic acid, phosphotidylcholine, poly-L-arginine, L-lysine, edetate disodium, citric acid, sodium citrate, sodium salicylate, chitosan and its related compounds, polyacrylic acid and its polymers, starch, chitosan, alkyl maltosides and its related compounds and/or combination thereof. The amount of the bioavailability enhancer may be present in the pharmaceutical composition in a range from about 0.1% w/w to about 15% w/w relative to the total weight of the composition.

Preservatives can be used to inhibit microbial growth in the compositions. Suitable preservatives which can be used, but not limited to, include benzalkonium chloride, benzethonium chloride, benzoic acid, benzyl alcohol, cetylpyridinium chloride, chlorobutanol, phenol, quaternary ammonium halides, phenylcarbinol, phenyl ethyl alcohol, phenylmercuric nitrate, phenylmercuric acetate, thimerosal, metacresol, myristylgamma picolinium chloride, potassium benzoate, potassium sorbate, sodium benzoate, sodium propionate, sorbic acid, thymol, and methyl, ethyl, propyl or butyl parabens, and other preservatives known to those of ordinary skill in the art. The amount of the preservative present in the intranasal pharmaceutical composition may range from about 0.005 to about 2% w/w relative to the total weight of the composition.

Cyclobenzaprine or salts thereof are stable when subjected to light and heat. However, they are very unstable towards oxidation of both the endocyclic and exocyclic double bonds to form epoxides as well as oxidation of the tertiary amine group to generate the N-oxide. In order to stabilize the intranasal pharmaceutical composition of cyclobenzaprine or salts thereof, antioxidants and/or chelating agent may be added.

Examples of suitable antioxidants for the composition according to the invention may be selected from, but not limited to, ascorbic acid, alpha-tocopherol (vitamin-E), butylated hydroxyanisole, butylated hydroxytoluene, glutathione, and the like. The amount of the antioxidant present in the pharmaceutical composition may range from about 0.0002 to about 2.0% w/w relative to the total weight of the composition.

Examples of suitable chelating agents for the composition according to the invention may be selected from, but not limited to, edetate disodium (EDTA), edetate trisodium, edetate tetrasodium, and diethyleneamine pentaacetate. The amount of the chelating agent present in the pharmaceutical composition may range from about 0.0001 to 2% w/w relative to the total weight of the composition.

Buffering agents are used to maintain the pH of the composition due to some external factors. Examples of suitable buffering agents for the composition according to the invention may be selected from, but are not limited to, borate buffers, tartrate buffers, lactate buffers, citrate buffers, phosphate buffers (e.g. potassium phosphate monobasic), citric acid/phosphate buffers, carbonate/carbonic acid buffers, succinate/succinic acid buffers, and tris(hydroxymethyl)aminomethane/hydrochloric acid buffers, disodium hydrogen phosphate, disodium hydrogen phosphate dodecahydrate dipotassium hydrogen phosphate, sodium carbonate, sodium bicarbonate, potassium dihydrogen phosphate, calcium carbonate, calcium bicarbonate, sodium dihydrogen phosphate, trisodium phosphate, tripotassium phosphate, potassium carbonate, potassium bicarbonate, potassium acetate, sodium acetate, dipotassium citrate, tripotassium citrate, disodium citrate, trisodium citrate, TRIS buffer, and other buffering agents known to those of ordinary skill in the art. The amount of buffering agent may range from about 0.005% w/w to about 2% w/w of the composition.

Examples of suitable mucoadhesive agent for the composition according to the invention may be selected from, but are not limited to, polyacrylic polymers, like carbopols, polycarbophil, carboxymethylcellulose or its pharmaceutically acceptable salts, microcrystalline cellulose, hydroxyethylcellulose, hydroxypropyl cellulose, hydroxypropylmethyl cellulose (i.e., hypromellose), methylcellulose, poloxamers, pectin, xanthan gums, alginates, gelatin alone, or in any combination thereof.

Examples of suitable pH adjusting agents for the composition according to the invention may be selected from, but not limited to, sodium hydroxide, hydrochloric acid, citric acid, acetic acid, fumaric acid, hydrochloric acid, malic acid, nitric acid, phosphoric acid, propionic acid, sulfuric acid, tartaric acid, or combinations thereof. The composition of the present invention comprises an amount of a pH adjusting agent sufficient to adjust the pH to about 5 to about 7. The amount of pH adjusting agent may range from about 0.005% w/w to about 2% w/w of the composition.

Tonicity agent is used to adjust the tonicity of the liquid formulation. Examples of suitable tonicity agent for the composition according to the invention may be selected from, but not limited to, one or more of magnesium chloride or sulphate; sodium bicarbonate, chloride and sulphate salts of lithium, sodium or potassium; sodium or potassium hydrogen phosphate, sodium or potassium acetate, magnesium succinate, sodium benzoate, sodium citrate, sodium ascorbate, arabinose, xylose, ribose, glucose, fructose, galactose, mannose, sucrose, maltose, trehalose, sorbitol, dextrose, raffinose, lactose, glycerin, glycine, leucine, alanine, methionine, sodium carboxy methyl cellulose, HPMC, hydroxyethyl methyl cellulose, crosslinked PVP, polyethylene oxide, carbopols, polyacrylamides, and other tonicity agents known to those of ordinary skill in the art. The amount of tonicity modifier may range from about 0.5% w/w to about 10% w/w of the composition.

Examples of suitable humectants may be selected from, but not limited to, propylene glycol, glycerin, butylene glycol, sorbitol, triacetin, and the like.

Examples of suitable sweetener/taste masking agents may be selected from, but not limited to, sucralose. thaumatin (e.g., Talin®) sucrose, saccharin (including the salt forms: sodium, calcium, etc.), fructose, glucose, dextrose, corn syrup, aspartame, acesulfame-K, xylitol, sorbitol, erythritol, ammonium glycyrrhizinate, thaumatin, neotame, mannitol, eucalyptus oil, camphor, and natural or artificial flavors or flavoring agents (for example menthol, mints, vanilla, orange, etc.), or combinations thereof.

Examples of suitable surfactants may be selected from, but not limited to, reaction products of natural or hydrogenated vegetable oils and ethylene glycol; i.e. polyoxyethylene glycolated natural or hydrogenated vegetable oils; for example, polyoxyethylene glycolated natural or hydrogenated castor oils, polyoxyl 35 castor oil, and polyoxyl 40 hydrogenated castor oil, Polyoxyethylene-sorbitan-fatty acid esters; e.g. lauryl, palmityl, stearyl and oleyl esters or the like. The amount of surfactant may range from about 0.0005% w/w to about 10% w/w of the composition.

In yet another embodiment, there is provided a method of treating or managing the muscle spasm in musculoskeletal diseases and/or painful physical symptoms in neurological diseases, the method comprising administering to a human being in need thereof, an intranasal pharmaceutical composition comprising from about 0.01 mg/dose to about 2.5 mg/dose of cyclobenzaprine or pharmaceutically acceptable salts thereof and one or more pharmaceutically acceptable excipients.

The musculoskeletal disease is selected from, but not limited to, the group caused by injuries and disorders that affect the human body's movement or musculoskeletal system (i.e. muscles, tendons, ligaments, nerves, discs, blood vessels, etc.).

The neurological diseases are disorders of the central and peripheral nervous system (i.e., the brain, spinal cord, cranial nerves, peripheral nerves, nerve roots, autonomic nervous system, neuromuscular junction, and muscles). These diseases involve the physical symptoms. Painful physical symptoms of neurological problems may include the partial or complete paralysis, muscle weakness, partial or complete loss of sensation, seizures, difficulty reading and writing, poor cognitive abilities, unexplained pain, and decreased alertness.

In another general aspect, the invention relates to a pharmaceutical kit comprising an intranasal pharmaceutical composition comprising from about 0.01 mg/dose to about 2.5 mg/dose of cyclobenzaprine or pharmaceutically acceptable salts thereof and one or more pharmaceutically acceptable excipients, and a nasal pump device for accurate delivery of said intranasal pharmaceutical composition.

The intranasal compositions of this invention are most effective when proper product delivery design is utilized. The preferred product design includes a composition of the invention contained within a delivery system, such as a bottle and a pump, for nasal delivery of the formulation in a mist of spray droplets to coat the mucosa of the nasal cavity upon administration. Preferred pumps for use in such products of the invention are metered multi-dose pumps; however, single unit-dose containers are also acceptable to deliver the therapeutic dose of cyclobenzaprine or salts thereof to the nasal cavity. The selection of the pump is based on the desired dose/spray volume and spray pattern appropriate for local delivery to the nasal mucosa. In certain such embodiments of the invention, the compositions can be effectively contained in a package comprising a bottle fitted with a screw cap, and are delivered by a metered-dose spray pump designed for intranasal application in volumes of 0.01 to 1.0 ml. Suitable compositions of the present invention include about 0.01 mg/dose to about 2.5 mg/dose of cyclobenzaprine or pharmaceutically acceptable salts thereof relative to the total weight of the composition.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### Example 1

Each 1 ml nasal spray contains:

| Sr. No. | Ingredient | mg/ml |
|---|---|---|
| 1 | Cyclobenzaprine Hydrochloride | 1.00 |
| 2 | Edetate Disodium | 0.50 |
| 3 | Sodium Chloride | 6.80 |
| 4 | Citric Acid Monohydrate | 0.50 |
| 5 | Disodium Phosphate Dihydrate | 3.13 |
| 6 | Hydroxypropylmethyl cellulose | 1.00 |
| 7 | Benzalkonium Chloride Solution (50%) | 0.25 |
| 8 | Water for Injection | q.s. to 1 mL |

### Example 2

Each 1 ml nasal spray contains:

| Sr. No. | Ingredient | mg/ml |
|---|---|---|
| 1 | Cyclobenzaprine Hydrochloride | 2.00 |
| 2 | Edetate Disodium | 0.50 |
| 3 | Sodium Chloride | 6.80 |
| 4 | Citric Acid Monohydrate | 0.50 |
| 5 | Disodium Phosphate Dihydrate | 3.13 |
| 6 | Hydroxypropylmethyl cellulose | 1.00 |
| 7 | Benzalkonium Chloride Solution (50%) | 0.25 |
| 8 | Water for Injection | q.s. to 1 mL |

### Example 3

Each 1 ml nasal spray contains:

| Sr. No. | Ingredient | mg/ml |
|---|---|---|
| 1 | Cyclobenzaprine Hydrochloride | 5.00 |
| 2 | Edetate Disodium | 0.50 |
| 3 | Sodium Chloride | 6.80 |
| 4 | Citric Acid Monohydrate | 0.50 |
| 5 | Disodium Phosphate Dihydrate | 3.13 |
| 6 | Hydroxypropylmethyl cellulose | 1.00 |
| 7 | Benzalkonium Chloride Solution (50%) | 0.25 |
| 8 | Water for Injection | q.s. to 1 mL |

### Example 4

Each 1 ml nasal spray contains:

| Sr. No. | Ingredient | mg/ml |
|---|---|---|
| 1 | Cyclobenzaprine Hydrochloride | 10.00 |
| 2 | Edetate Disodium | 0.50 |
| 3 | Sodium Chloride | 6.80 |
| 4 | Citric Acid Monohydrate | 0.50 |
| 5 | Disodium Phosphate Dihydrate | 3.13 |
| 6 | Hydroxypropylmethyl cellulose | 1.00 |
| 7 | Benzalkonium Chloride Solution (50%) | 0.25 |
| 8 | Water for Injection | q.s. to 1 mL |

### Example 5

Each 1 ml nasal spray contains:

| Sr. No. | Ingredient | mg/ml |
|---|---|---|
| 1 | Cyclobenzaprine Hydrochloride | 20.00 |
| 2 | Edetate Disodium | 0.50 |
| 3 | Sodium Chloride | 6.80 |
| 4 | Citric Acid Monohydrate | 0.50 |
| 5 | Disodium Phosphate Dihydrate | 3.13 |
| 6 | Hydroxypropylmethyl cellulose | 1.00 |
| 7 | Benzalkonium Chloride Solution (50%) | 0.25 |
| 8 | Water for Injection | q.s. to 1 mL |

Process:

1) Citric acid monohydrate, disodium phosphate dihydrate, edetate disodium, sodium chloride, benzalkonium chloride solution (50%), hydroxypropylmethyl cellulose and water for injection were mixed together under continuous stirring;

2) Cyclobenzaprine was added in the solution of step 1 under continuous stirring and final weight was made up with water for injection; and

3) The solution of step 2 was filtered through a 0.22 μm filter and filled in a suitable container closure system.

### Example 6

Each 1 ml nasal spray contains:

| Sr. No. | Ingredient | mg/ml |
|---|---|---|
| 1 | Cyclobenzaprine Hydrochloride | 5.00 |
| 2 | Edetate Disodium | 0.50 |
| 3 | Sodium Chloride | 6.80 |
| 4 | Citric Acid Monohydrate | 0.50 |
| 5 | Disodium Phosphate Dodecahydrate | 6.30 |
| 6 | Hydroxypropylmethyl cellulose | 1.00 |
| 7 | Benzalkonium Chloride Solution (50%) | 0.25 |
| 8 | Water for Injection | q.s. to 1 mL |

Process:

1) Citric acid monohydrate, disodium phosphate dodecahydrate, edetate disodium, sodium chloride, benzalkonium chloride solution (50%), hydroxypropylmethyl cellulose and water for injection were mixed together under continuous stirring;

2) Cyclobenzaprine was added in the solution of step 1 under continuous stirring and final weight was made up with water for injection; and

3) The solution of step 2 was filtered through a 0.22 μm filter and filled in a suitable container closure system.

### Example 7

| | | Quantity |
|---|---|---|
| Sr. No. | Name of Ingredients | mg/mL |
| 1 | Cyclobenzaprine HCL | 1.75 |
| 2 | Benzalkonium chloride 50% solution | 0.40 |
| 3 | Phenyl ethyl alcohol | 2.50 |
| 4 | Dextrose anhydrous | 50.00 |
| 5 | Hydroxypropylmethyl cellulose | 1.00 |
| 6 | Polyoxyethylene sorbitan ester | 0.05 |
| 7 | Water for Injection | qs to 1 mL |

Process:

1. Approximately 80% of water for injection was collected in a clean stainless steel (SS) vessel.

2. Benzalkonium chloride 50% solution was added in water for injection under continuous stirring until clear solution was achieved.

3. Dextrose Anhydrous was added in the solution of step-2 under continuous stirring until clear solution was achieved.

4. Polyoxyethylene sorbitan esters was added in the solution of step-3 under continuous stirring until clear solution was achieved.

5. Phenyl ethyl alcohol was added in the solution of step-4 under continuous stirring until clear solution achieved.

6. Hydroxypropylmethyl cellulose was dissolved in the water for injection having temperature in range of 50° C. to 60° C. under stirring to form clear solution. This solution was kept for cooling at room temperature.

7. Solution of step-6 was mixed with the solution of the step-5 solution under stirring to form uniform solution.

8. Cyclobenzaprine hydrochloride was dissolved in the solution of step-7 under stirring to form a clear solution.

9. The solution of step 8 was filtered through a 0.22 μm filter and filled in a suitable container closure system.

Preclinical Study-1:

Objective: To assess in-vivo absorption of cyclobenzaprine formulation following oral (PO) and intranasal (IN) administration in male wistar rats.

Method: Male wistar rats (n=4) received 0.40 mg/kg of cyclobenzaprine via intranasal administration under non-fasted conditions. Male wistar rats (n=5) received 1.05 mg/kg of cyclobenzaprine via oral route. A serial blood sampling was performed from each animal. The blood samples were collected at 0.00 (pre-dose), 0.083, 0.25, 0.5, 1, 2, 4, 6, 8, 24 hrs. Plasma concentrations of Cyclobenzaprine were determined using a sensitive and specific fit-for-purpose liquid chromatographic tandem mass spectrometry (LC-MS/MS) method. Pharmacokinetic parameters were calculated using the non-compartmental analysis (NCA) module of Phoenix-WinNonlin® software version 6.4.

Results:

| Study | Oral | Intranasal |
|---|---|---|
| Dose (mg/kg) | 1.05 | 0.4 |
| Tₘₐₓ (Hour) | 0.5 (0.25-1.0) | 0.083 |
| Cₘₐₓ (ng/ml) | 1.74 ± 0.95 | 31.27 ± 10.14 |
| AUCₗₐₛₜ (ng*h/mL) | 2.98 ± 0.42 | 51.20 ± 12.83 |
| AUC_{inf} (ng*h/mL) | 3.27 ± 0.42 | 52.34 ± 12.76 |
| T_{1/2} (hour) | 1.43 ± 0.33 | 2.79 ± 2.35 |
| DNAUC* | 3.11 | 130.85 |
| Fold increase in IN | — | 42 |
| absorption | | |

*Tₘₐₓ reported as median (range);
*DNAUC, AUC normalized at 1 mg/kg dose

Observations: After intranasal administration, nasal composition showed much higher plasma cyclobenzaprine levels and faster onset of absorption relative to the oral cyclobenzaprine immediate release oral dosage form. Nasal composition provided 42 fold increase in the absorption in comparison to oral dosage form.

Preclinical Study-2:

Objective: To investigate the pharmacokinetic parameters of cyclobenzaprine following single intranasal administration vs. oral administration in male beagle dogs.

Method: Male beagle dogs (n=4) received 0.26 mg/kg of cyclobenzaprine via intranasal administration under non-fasted conditions. Male beagle dogs (n=4) received 0.51 mg/kg (Equivalent to 5 mg tablet) of cyclobenzaprine via oral route. A serial blood sampling was performed from each animal. The blood samples were collected at 0.00 (pre-dose), 0.083, 0.25, 0.5, 1, 2, 4, 6, 8, 24 hrs. Plasma concentrations of Cyclobenzaprine were determined using a sensitive and specific fit-for-purpose liquid chromatographic tandem mass spectrometry (LC-MS/MS) method. Pharmacokinetic parameters were calculated using the non-compartmental analysis (NCA) module of Phoenix-WinNonlin® software version 6.4.

Results:

| | Oral | Intranasal |
|---|---|---|
| Study | administration | administration |
| Dose (mg/kg) | 0.51 | 0.26 |
| Tmax (h) | 1.5 (0.5-2.0) | 0.083 (0.083-0.25) |
| Cₘₐₓ (ng/mL) | 1.61 ± 1.21 | 62.29 ± 13.83 |
| AUCₗₐₛₜ (ng*h/mL) | 7.03 ± 5.65 | 100.34 ± 25.92 |
| AUC_{inf} (ng*h/mL) | 7.58 ± 5.64 | 103.14 ± 26.20 |
| T_{1/2} (h) | 2.03 ± 0.64 | 6.09 ± 2.27 |
| DNAUC* | 13.52 | 385.92 |
| Fold increase in IN | — | 28.5 |
| absorption | | |

*DNAUC, AUC normalized at 1 mg/kg dose.

Observations: After intranasal administration, nasal composition showed much higher plasma cyclobenzaprine levels and faster onset of absorption relative to the oral cyclobenzaprine immediate release oral dosage form. Nasal composition provided 28.5 fold increase in the absorption to oral dosage form.

Preclinical Study-3:

Objective: To evaluate the comparative pharmacokinetic parameters of cyclobenzaprine between intranasal dose and oral dose in male wistar rat.

Method: Male wistar rats (n=4) received 0.02 mg/kg of cyclobenzaprine via intranasal administration under non-fasted conditions. Male wistar rats (n=4) received 0.885 mg/kg of cyclobenzaprine via oral route. A serial blood sampling was performed from each animal. The blood samples were collected at 0.00 (pre-dose), 0.08, 0.25, 0.5, 1, 2, 4, 6, 8, 24 hrs. Plasma concentrations of Cyclobenzaprine were determined using a sensitive and specific fit-for-purpose liquid chromatographic tandem mass spectrometry (LC-MS/MS) method. Pharmacokinetic parameters were calculated using the non-compartmental analysis (NCA) module of Phoenix-WinNonlin® software version 6.4.

Results:

| | Oral | Intranasal |
|---|---|---|
| Study | administration | administration |
| Dose (mg/kg) | 0.885 | 0.020 |
| Tₘₐₓ (h) | 1.00 | 0.083 |
| | (0.50-1.00) | |
| Cₘₐₓ (ng/mL) | 1.200 | 2.835 |
| | (0.388-1.866) | (1.986-3.983) |
| AUCₗₐₛₜ (ng*h/mL) | 2.538 | 2.321 |
| | (1.454-4.025) | (1.73-2.854) |
| AUC_{inf} (ng*h/mL) | 3.262 | 2.587 |
| | (2.567-4.661) | (2.151-3.142) |
| T_{1/2} (h) | 2.614 | 1.075 |
| | (1.475-5.01) | (0.864-1.232) |

Observations:

After intranasal administration, nasal composition showed the faster rate of absorption, the higher plasma concentration at Tₘₐₓ and comparable plasma concentration over a time to the oral cyclobenzaprine immediate release dosage form.

Preclinical Study-4

Objective: To assess in vivo absorption of cyclobenzaprine formulation following oral (PO) and intranasal (IN) administration in New Zealand white rabbit.

Method: New Zealand white rabbit (n=4) received 0.011 mg/kg of cyclobenzaprine via intranasal administration under fed conditions. New Zealand white rabbit (n=4) received 0.442 mg/kg of cyclobenzaprine via oral route. A serial blood sampling was performed from each animal. The blood samples were collected at 0.00 (pre-dose), 0.083, 0.25, 0.5, 1, 2, 4, 6, 8, 24 hrs. Plasma concentrations of cyclobenzaprine were determined using a sensitive and specific fit-for-purpose liquid chromatographic tandem mass spectrometry (LC-MS/MS) method. Pharmacokinetic parameters were calculated using the non-compartmental analysis (NCA) module of Phoenix-WinNonlin® software version 6.4.

Results:

| | | Intranasal | Oral |
|---|---|---|---|
| Study Parameters | | 0.011 mg/kg | 0.442 mg/kg |
| Tₘₐₓ | (h) | 0.29 | Plasma concentrations |
| Cₘₐₓ | (ng/mL) | 0.56 | were found below limit |
| AUCₗₐₛₜ | (h*ng/mL) | 0.52 | of quantification; hence |
| AUC_{INF}_{_{—}}_{pred} | (h*ng/mL) | 0.92 | PK parameter was not |
| V_{z}_{_{—}}_{F}_{_{—}}_{pred} | (L/kg) | 28.55 | calculated. |
| Cₗ_{_{—}}_{F}_{_{—}}_{pred} | (mL/min/kg) | 199.56 | |
| H_{L}_{_{—}}_{Lambda}_{_{—}}_{z} | (h) | 1.65 | |
| MRTₗₐₛₜ | (h) | 0.87 | |
| MRT_{INF}_{_{—}}_{pred} | (h) | 2.43 | |

Observations: After intranasal administration, nasal composition showed significant plasma concentration at a very low dose. On the other hand, plasma concentrations were found below the limit of quantification after oral administration in rabbits.

Preclinical Study-5:

Objective: To determine pharmacokinetic profile of cyclobenzaprine following intranasal administration

Method Parameters:

| | |
|---|---|
| Test product | Cyclobenzaprine |
| Formulation strength | 0.25% w/v |
| Animal model | Wistar male rats |
| No of animals | n = 4/dose group |
| Route of administration | Intranasal |
| Dose (mg/kg) | 0.05, 0.1 and 0.2 mg/kg |
| Dose Volume | 5 μL, 10 μL and 10 μL + 10 μL per |
| | rat |
| Fasting/Non-Fasting | Fed |
| Blood sampling time point | Predose, 0.083, 0.25, 0.5, 1.0, 2.0, 4.0, |
| | 6.0, 8.0, 24.0 hour |
| Analyte to be measured | Cyclobenzaprine |
| Method | LC-MS/MS |
| CC range/Sensitivity | 0.050 to 10 ng/mL |

Results:

| Study | Intranasal | | | | |
|---|---|---|---|---|---|
| Parameters/dose | 0.05 | mg/kg | 0.1 | mg/kg | 0.2 mg/kg |
| planned | | | | | |
| Actual Dose, mg/kg | 0.03 | mg/kg | 0.07 | mg/kg | 0.14 mg/kg |
| Dose volume | 5 | μL | 10 | μL | 10 μL + 10 μL^{a} |

| | | | |
|---|---|---|---|
| Tₘₐₓ (h) | 0.083 | 0.083 | 0.083 |
| Cₘₐₓ (ng/mL) | 4.6 ± 1.7 | 16.4 ± 11.6 | 27.6 ± 26.4 |
| AUC₍₀₋ₜ₎ (ng · h/mL) | 4.8 ± 1.4 | 10.6 ± 3.0 | 22.4 ± 10.1 |
| T½ (h) | 1.9 ± 0.3 | 1.6 ± 0.2 | 2.2 ± 0.3 |

Tmax reported as median, Mean ± SD, N = 4,
^{a}Administered 10 μL in each nostril, total 20 μL/rat

Observations:

1. Dose dependent linear bioavailability was observed. With increasing the dose, the bioavailability was also increased.

2. Time to reach the maximum concentration (Tₘₐₓ) was observed to be same for all doses.

While the invention has been described in terms of its specific embodiments, certain modifications and equivalents will be apparent to those skilled in the art and are intended to be included within the scope of the invention.

1-17. (canceled)

18. An intranasal pharmaceutical composition comprising from about 0.01 mg/dose to about 2.5 mg/dose of cyclobenzaprine or pharmaceutically acceptable salts thereof and one or more pharmaceutically acceptable excipients.

19. The intranasal pharmaceutical composition of claim 18, wherein the intranasal pharmaceutical composition provides a Cₘₐₓ of cyclobenzaprine or salts thereof from about 5 ng/mL to about 100 ng/mL at a tₘₐₓ of less than 120 minutes after administration.

20. The intranasal pharmaceutical composition of claim 19, wherein the intranasal pharmaceutical composition provides a Cₘₐₓ of cyclobenzaprine or salts thereof from about 5 ng/mL to about 100 ng/mL at a tₘₐₓ of less than 60 minutes after administration.

21. The intranasal pharmaceutical composition of claim 19, wherein the intranasal pharmaceutical composition provides a Cₘₐₓ of cyclobenzaprine or salts thereof from about 5 ng/mL to about 100 ng/mL at a tₘₐₓ of less than 30 minutes after administration.

22. The intranasal pharmaceutical composition of claim 18, wherein the intranasal composition has a pH of from about 5 to about 7.

23. The intranasal pharmaceutical composition of claim 18, wherein the pharmaceutically acceptable excipients comprise one or more of preservatives, antioxidants, buffering agents, mucoadhesive agents, acidifying agents, alkalizing agents, sweeteners, taste masking agents, humectants, chelating agents, tonicity agents, surfactants, and/or bioavailability enhancers.

24. The intranasal pharmaceutical composition of claim 23, wherein the preservatives comprise from about 0.005 to about 2% w/w of the composition.

25. The intranasal pharmaceutical composition of claim 23, wherein the surfactants comprise from about 0.0005% w/w to about 10% w/w of the composition.

26. The intranasal pharmaceutical composition of claim 18, wherein the pharmaceutical composition is prepared by a process comprising:
i) mixing one or more pharmaceutically acceptable excipients with water for injection under continuous stirring to form a solution;
ii) dissolving cyclobenzaprine or pharmaceutically acceptable salts thereof into the solution of step i) under continuous stirring; and
iii) filtering the solution of step ii) through a microfilter and packed in a suitable container.

27. A method of treating or managing muscle spasm in musculoskeletal diseases and/or painful physical symptoms in neurological diseases, the method comprising administering to a human being in need thereof, an intranasal pharmaceutical composition comprising from about 0.01 mg/dose to about 2.5 mg/dose of cyclobenzaprine or pharmaceutically acceptable salts thereof and one or more pharmaceutically acceptable excipients.

28. The method of claim 27, wherein the intranasal pharmaceutical composition is administered via a nasal pump device.

29. The method of claim 28, wherein the nasal pump device is a metered dose nasal pump device or a unit dose nasal pump device.

30. The method of claim 28, wherein the nasal pump device is designed for intranasal application in volumes of 0.01 to 1.0 ml per administration.

31. A pharmaceutical kit comprising an intranasal pharmaceutical composition comprising from about 0.01 mg/dose to about 2.5 mg/dose of cyclobenzaprine or pharmaceutically acceptable salts thereof and one or more pharmaceutically acceptable excipients, and a nasal pump device.

32. The pharmaceutical kit of claim 31, wherein the composition is deliverable via a metered dose nasal pump device or a unit dose nasal pump device.

33. The pharmaceutical kit of claim 31, wherein the nasal pump device is designed for intranasal application in volumes of 0.01 to 1.0 ml per administration.
